(19) United States
(12) Patent Application Publication
(10) Pub. No.: US 2023/0346383 A1
(43) Pub. Date: Nov. 2, 2023

(54) SYSTEMS AND METHODS FOR TREATING HEART FAILURE BY REDIRECTING BLOOD FLOW IN THE AZYGOS VEIN

(71) Applicant: inQB8 Medical Technologies, LLC, Winchester, MA (US)

(72) Inventors: Arshad Quadri, West Hartford, CT (US); J. Brent Ratz, Winchester, MA (US); Christopher William Stivers, Somerville, MA (US)

(21) Appl. No.: 18/300,293

(22) Filed: Apr. 13, 2023

Related U.S. Application Data
(60) Provisional application No. 63/331,496, filed on Apr. 15, 2022.

(51) Int. Cl. A61B 17/12

(52) U.S. Cl. CPC A61B 17/12118; A61B 17/12036

(57) ABSTRACT

Systems and methods for treating heart failure by redirecting blood flow in the azygos vein are disclosed, as well as systems, devices, and methods for controllably and selectively occluding, restricting, and/or diverting flow within a patient’s vasculature. Systems, devices and methods that redirect blood flow in the azygos vein. Devices may include an implant configured to redirect blood from a pulmonary artery to an azygos vein, and an implant configured to be positioned in an azygos vein of a patient to at least partially occlude blood flow from the azygos vein into a superior vena cava.

### INCORPORATION BY REFERENCE TO ANY PRIORITY APPLICATIONS

This application claims the benefit of priority under 35 U.S.C. § 119(e) to U.S. Provisional Pat. Application No 63/331,496, filed Apr. 15, 2022. All of the above-mentioned applications are hereby incorporated by reference herein in their entireties. Any and all applications for which a foreign or domestic priority claim is identified in the Application Data Sheet as filed with the present application are hereby incorporated by reference under 37 CFR 1.57.

### TECHNICAL FIELD

The present disclosure relates to systems, devices, and methods for treating heart failure, as well as systems, devices, and methods for controllably and selectively occluding, restricting, and/or diverting flow within a patient’s vasculature, including systems, devices and methods that redirect blood flow in the azygos vein.

### BACKGROUND

### Field of the Invention

Heart failure, or congestive heart failure, can occur when the heart fails to efficiently pump blood. Certain cardiovascular conditions, including narrowed arteries in the heart and high blood pressure, can gradually weaken and/or stiffen the heart muscle, reducing cardiac efficiency. As cardiac output decreases, blood pressure drops and blood circulation slows. Fluid can build up in the lungs, causing shortness of breath. Reduced blood flow and increased fluid further compromise the heart, and can eventually become life-threatening.

At the same time, the kidneys experience a drop in renal blood pressure as the heart begins to fail. In response, renal sympathetic activation releases hormones, including renin, angiotensin, and aldosterone to increase water and sodium retention as well as increase extracellular fluid in an attempt to raise the blood pressure and drive blood back to the heart. The increased pressure and fluid exacerbate the stress on the compromised heart, further accelerating the failure mechanism.

Heart failure can be initially treated by lifestyle changes to reduce the load on the heart, but in many cases, medication and/or renal intervention (such as ablation) may be used to reduce blood pressure and fluid buildup. As cases progress in severity, surgical intervention, such as coronary bypass, stent placement, heart valve repair or replacement, implantation of cardioverter-defibrillator, use of ventricular assist device, or heart transplant, may become necessary.

### SUMMARY

Certain aspects of the present application are directed to methods, systems and devices for treating heart failure. Certain aspects of the present application are directed to methods, systems and devices that divert or redirect blood flow in the azygos vein.

In some aspects, the techniques described herein relate to a system for treating heart failure of a patient, including: an implant configured to be positioned in an azygos vein of a patient to at least partially occlude blood flow from the azygos vein into a superior vena cava. In some aspects, the implant further includes a controllable valve. In some aspects, the implant includes a shunt configured to be positioned between a pulmonary artery and the azygos vein.

In some aspects, the techniques described herein relate to a system, wherein the implant is configured to redirect blood in the azygos vein. In some aspects, the implant is configured to divert blood in the azygos vein into a hemiazygos vein, an accessory hemiazygos vein, an internal mammary or internal thoracic vein.

In some aspects, the techniques described herein relate to a system, wherein the implant includes an expandable tubular body configured to extend between a pulmonary artery and the azygos vein. In some aspects, the implant includes an upstream end that does not substantially obstruct blood flow in the pulmonary artery. In some aspects, the implant includes an upstream end configured to radially expand against an inner wall of the pulmonary artery. In some aspects, the implant includes a downstream end configured to radially expand against an inner wall of the azygos vein. In some aspects, the downstream end of the implant is configured to direct blood into the azygos vein opposite to a forward direction of blood flow in the azygos vein.

In some aspects, the techniques described herein relate to a system, wherein the implant includes a valve configured to regulate blood flow from the azygos vein into a superior vena cava. In some aspects, the implant includes a porous section configured to permit blood flow from the azygos vein into a superior vena cava.

In some aspects, the techniques described herein relate to a system, wherein the implant includes a first implant configured to be positioned in the azygos vein to at least partially occlude blood flow from the azygos vein into the superior vena cava, and further including a second implant configured to be direct blood from a pulmonary artery into the azygos vein.

In some aspects, the techniques described herein relate to a system, further including a controller configured to regulate blood flow through the implant. In some aspects, the controller is configured to regulate blood flow through the implant based on one or more pressure readings.

In some aspects, the techniques described herein relate to a method of treating heart failure of patient, including restricting blood from flowing from an azygous vein into a superior vena cava of the patient. In some aspects, the blood is restricted from flowing from the azygos vein into the superior vena cava by an implant positioned in the azygos vein that at least partially occludes blood flow in the azygous vein. In some aspects, the implant is chronically implanted.

In some aspects, the techniques described herein relate to a method, wherein the blood is restricted from flowing from the azygos vein into the superior vena cava by positioning a shunt between a pulmonary artery and the azygos vein that diverts blood from the pulmonary artery into the azygous vein. In some aspects, the shunt is configured to direct blood into the azygous vein against a direction of blood flow within the azygous vein. In some aspects, the shunt does not extend substantially into the pulmonary artery or into the azygos vein. In some aspects, the shunt has a length that extends within the pulmonary artery and within the azygos vein.

In some aspects, the techniques described herein relate to a method, wherein the shunt has an upstream end within the pulmonary artery that is upstream of a connection between the pulmonary artery and the azygos vein with respect to a direction of blood flow within the pulmonary artery. In some aspects, the shunt has a downstream end within the azygos vein that is upstream of the connection between the pulmonary artery and the azygous vein with respect to a direction of blood flow within the azygos vein. In some aspects, the shunt is positioned between a superior right pulmonary artery and the azygos vein.

In some aspects, the techniques described herein relate to a method for treating heart failure of a patient, the method including diverting blood from a pulmonary artery to an azygos vein via a shunt implanted between the pulmonary artery and the azygos vein. In some aspects, the diverting of blood is sufficient to decongest lungs of the patient and reduce a left ventricular end diastolic pressure (LVEDP). In some aspects, the diverting of blood is sufficient to reduce pulmonary artery pressure to relieve pulmonary hypertension and consequently reduce a workload of a right ventricle of the patient. In some aspects, the diverting of blood is sufficient to mimic a splanchnic vascular capacitance and redistribute blood into a splanchnic compartment of the patient. In some aspects, the diverting of blood is sufficient to cause dilation and/or increased pressure within intercostal veins of the patient. In some aspects, the diverting of blood causes a redirection of blood from the pulmonary artery into the azygos vein and into a superior vena cava. In some aspects, the diverting of blood causes a redirection of blood from the pulmonary artery into the azygos vein and into a hemiazygos vein. In some aspects, the diverting of blood causes a redirection of blood from the pulmonary artery into the azygos vein and into an accessory hemiazygos vein. In some aspects, the diverting of blood causes a redirection of blood from the pulmonary artery into the azygos vein and into an internal mammary or internal thoracic vein.

In some aspects, the techniques described herein relate to a method, wherein the blood is diverted from a right pulmonary artery to the azygos vein. In some aspects, the techniques described herein relate to a method, further including restricting and/or occluding blood from flowing from the azygos vein into a superior vena cava.

In some aspects, the techniques described herein relate to a method, wherein an implantable shunt includes a controllable valve. In some aspects, the techniques described herein relate to a method, further including controlling an amount of blood that flows through the shunt with a controller. In some aspects, the techniques described herein relate to a method, further including controlling an amount of blood that flows through the shunt based on feedback received from one or more pressure sensors positioned within the patient.

In some aspects, the techniques described herein relate to a method, wherein the shunt is implanted between the pulmonary artery and the azygos vein by: delivering a first catheter within the pulmonary artery; delivering a second catheter within the azygos vein; aligning a first magnet carried by the first catheter with a second magnet carried by the second catheter to align the first and the second catheters; delivering a guidewire between the pulmonary artery and the azygos vein while the first and second catheters are aligned; and using the guidewire to deliver the shunt or a delivery device for the shunt between the pulmonary artery and the azygos vein.

In some aspects, the techniques described herein relate to an implantable shunt configured to be implanted between a pulmonary artery and an azygos vein of a patient and configured to divert flow into the azygos vein for treatment of heart failure.

In some aspects, the techniques described herein relate to an implantable flow control system, including: an implant including: an expandable body including a proximal end and a distal end and a lumen extending from the proximal end to the distal end, wherein the expandable body is configured to collapse to a collapsed configuration for delivery into a patient and to expand from the collapsed configuration to an expanded configuration for implantation within the patient; and a fluid restrictor positioned within the expandable body, the fluid restrictor including: a first partition partially blocking a first portion of the lumen when the expandable body is in the expanded configuration; and a second partition moveable relative to the first partition to selectively block and unblock a second portion of the lumen when the expandable body is in the expanded configuration.

In some aspects, the techniques described herein relate to a system, wherein the first partition includes a first expandable wire frame and a first fabric portion extending across the first expandable wire frame, and the second partition includes a second expandable wire frame and a second fabric portion extending across the second expandable wire frame.

In some aspects, the second partition is configured to rotate relative to the first partition to selectively increase or decrease a size of an opening through the lumen. In some aspects, the techniques described herein relate to a system, wherein the first partition is fixed relative to the expandable body. In some aspects, a controller is configured to control movement of the second partition relative to the first partition.

In some aspects, the techniques described herein relate to a system, wherein the implant includes a shunt configured to be implanted between a first vessel and a second vessel of the patient. In some aspects, t the implant is configured to be expanded within a vessel of the patient.

In some aspects, the techniques described herein relate to a system for treating heart failure, including: an implantable shunt configured to divert blood flow within a patient from a first vessel to a second vessel, wherein the implantable shunt is implantable between the first vessel and the second vessel, and wherein the implantable shunt includes an adjustable flow opening; and a controller configured to control the adjustable flow opening to control an amount of blood that flows through the implantable shunt, wherein the controller is configured to receive readings from one or more pressure sensors positioned within the patient, and wherein the controller is programmed to control the adjustable flow opening based on the readings.

In some aspects, the first vessel is a right pulmonary artery and the second vessel is an azygos vein. In some aspects, the controller is configured to receive one or more of a central venous pressure, a right ventricular pressure, a pulmonary artery pressure, an aortic, and a left atrial pressure.

In some aspects, the techniques described herein relate to an implantable shunt including one or more features of the foregoing description. In some aspects, the techniques described herein relate to a method including one or more features of the foregoing description. In some aspects, the techniques described herein relate to a system including one or more features of the foregoing description.

Certain aspects of the present application are directed to methods, systems and devices for creating a shunt between two blood vessels, such as between the pulmonary artery and the azygos vein, to selectively divert or control blood flow. In one aspect, a method for treating heart failure of a patient is provided. The method comprises diverting blood from a pulmonary artery to an azygos vein via a shunt implanted between the pulmonary artery and the azygos vein.

The methods, systems and devices described above or as described further herein may further comprise one or more of the following features. The diverting of blood may be sufficient to decongest lungs of the patient and reduce a left ventricular end diastolic pressure (LVEDP). The diverting of blood may be sufficient to reduce pulmonary artery pressure to relieve pulmonary hypertension and consequently reduce a workload of a right ventricle of the patient. The diverting of blood may be sufficient to mimic a splanchnic vascular capacitance and redistribute blood into a splanchnic compartment of the patient. The diverting of blood may be sufficient to cause dilation and/or increased pressure within intercostal veins of the patient. The diverting of blood may cause a redirection of blood from the pulmonary artery into the azygos vein and into a superior vena cava. The diverting of blood may cause a redirection of blood from the pulmonary artery into the azygos vein and into a hemiazygos vein. The diverting of blood may cause a redirection of blood from the pulmonary artery into the azygos vein and into an accessory hemiazygos vein. The diverting of blood may cause a redirection of blood from the pulmonary artery into the azygos vein and into an internal mammary or internal thoracic vein. The blood may be diverted from a right pulmonary artery to the azygos vein.

The method, systems and devices described above or described further herein may comprise restricting and/or occluding blood from flowing from the azygos vein into a superior vena cava. In embodiments incorporating a shunt, the shunt may comprise a controllable valve. The methods, systems and devices may further comprise controlling an amount of blood that flows through the shunt with a controller. The methods, systems and devices may further comprise controlling an amount of blood that flows through the shunt based on feedback received from one or more pressure sensors positioned within the patient. The shunt may be implanted between the pulmonary artery and the azygos vein by delivering a first catheter within the pulmonary artery, delivering a second catheter within the azygos vein, aligning a first magnet carried by the first catheter with a second magnet carried by the second catheter to align the first and the second catheters, delivering a guidewire between the pulmonary artery and the azygos vein while the first and second catheters are aligned, and using the guidewire to deliver the shunt or a delivery device for the shunt between the pulmonary artery and the azygos vein.

In one aspect, an implantable shunt is provided configured to be implanted between a pulmonary artery and an azygos vein of a patient and configured to divert flow into the azygos vein for treatment of heart failure. The implantable shunt may be configured to operate or be implanted according to any of the methods described above or as described further herein.

In one aspect, an implantable flow control system is provided. The system may comprise an implant comprising an expandable body comprising a proximal end and a distal end and a lumen extending from the proximal end to the distal end, wherein the expandable body is configured to collapse to a collapsed configuration for delivery into a patient and to expand from the collapsed configuration to an expanded configuration for implantation within the patient. The system may further comprise a fluid restrictor positioned within the expandable body. The fluid restrictor may comprise a first partition partially blocking a first portion of the lumen when the expandable body is in the expanded configuration, and a second partition moveable relative to the first partition to selectively block and unblock a second portion of the lumen when the expandable body is in the expanded configuration.

The system described above or as described further herein may further comprise one or more of the following features. The first partition may comprise a first expandable wire frame and a first fabric portion extending across the first expandable wire frame, and the second partition may comprise a second expandable wire frame and a second fabric portion extending across the second expandable wire frame. The second partition may be configured to rotate relative to the first partition to selectively increase or decrease a size of an opening through the lumen. The first partition may be fixed relative to the expandable body. The system may further comprise a controller configured to control movement of the second partition relative to the first partition. The implant may further comprise a shunt configured to be implanted between a first vessel and a second vessel of the patient. The implant may be configured to be expanded within a vessel of the patient.

In one aspect, a system for treating heart failure is provided. The system may comprise an implantable shunt configured to divert blood flow within a patient from a first vessel to a second vessel, wherein the implantable shunt is implantable between the first vessel and the second vessel, and wherein the implantable shunt comprises an adjustable flow opening. The system may further comprise a controller configured to control the adjustable flow opening to control an amount of blood that flows through the implantable shunt, wherein the controller is configured to receive readings from one or more pressure sensors positioned within the patient, and wherein the controller is programmed to control the adjustable flow opening based on the readings.

The system described above or as described further herein may further comprise one or more of the following features. The first vessel may be a right pulmonary artery and the second vessel may be an azygos vein. The controller may be configured to receive one or more of a central venous pressure, a right ventricular pressure, a pulmonary artery pressure, an aortic, and a left atrial pressure.

### BRIEF DESCRIPTION OF THE DRAWINGS

Certain features of this disclosure are described below with reference to the drawings. The illustrated implementations are intended to illustrate, but not to limit, the implementations. Various features of the different disclosed implementations can be combined to form further implementations, which are part of this disclosure.

FIG. 1A illustrates cardiovascular features near the heart.

FIG. 1B illustrates the venous system in the chest.

FIG. 1C illustrates relevant anatomic structures in the upper chest.

FIG. 2A illustrates another view of relevant anatomic structures in the upper chest.

FIG. 2B illustrates an example of an implant location relative to the view shown in FIG. 2A.

FIG. 3A illustrates the pulmonary artery and azygos vein blood flow before intervention.

FIG. 3B illustrates an example of the blood flow of FIG. 3A after shunt implantation.

FIGS. 4A-4C illustrate various views of one implementation of a shunt implant.

FIGS. 5A and 5B illustrate one example of the altered blood flow path in the pulmonary artery and azygos vein after implantation of an implementation of the device.

FIG. 6A illustrates an implementation of a sleeve implant and resulting blood flow.

FIG. 6B illustrates an implementation of a valved implant and resulting blood flow.

FIG. 6C illustrates an implementation of a porous implant and resulting blood flow.

FIG. 7 illustrates an example on an implementation for obtaining access between the pulmonary artery and the azygos vein.

FIGS. 8A and 8B illustrate an implementation of a controllable valve that may be utilized in an implant.

FIG. 9 illustrates an implementation of a shunt implant with a controllable valve.

FIG. 10 illustrates an implementation of a system with an adjustable shunt.

### DETAILED DESCRIPTION

Various features and advantages of this disclosure will now be described with reference to the accompanying figures. The following description is merely illustrative in nature and is in no way intended to limit the disclosure, its application, or uses. This disclosure extends beyond the specifically disclosed implementations and/or uses and obvious modifications and equivalents thereof. Thus, it is intended that the scope of this disclosure should not be limited by any particular implementations described below. The features of the illustrated implementations can be modified, combined, removed, and/or substituted as will be apparent to those of ordinary skill in the art upon consideration of the principles disclosed herein. Furthermore, implementations disclosed herein can include several novel features, no single one of which is solely responsible for its desirable attributes or which is essential to practicing the systems, devices, and/or methods disclosed herein.

Parts, components, features, and/or elements of the systems and devices described herein that can function the same or similarly across various implementations are identified using similar reference numerals. Differences between the various implementations are discussed herein.

Implementations of the present application relate to controlling cardiac output to treat or prevent heart failure in patients. Certain embodiments are directed to creating a shunt (which may also be referred to as an implant, conduit or arteriovenous (AV) fistula) between the right pulmonary artery and the azygos vein to divert blood from the right pulmonary artery.

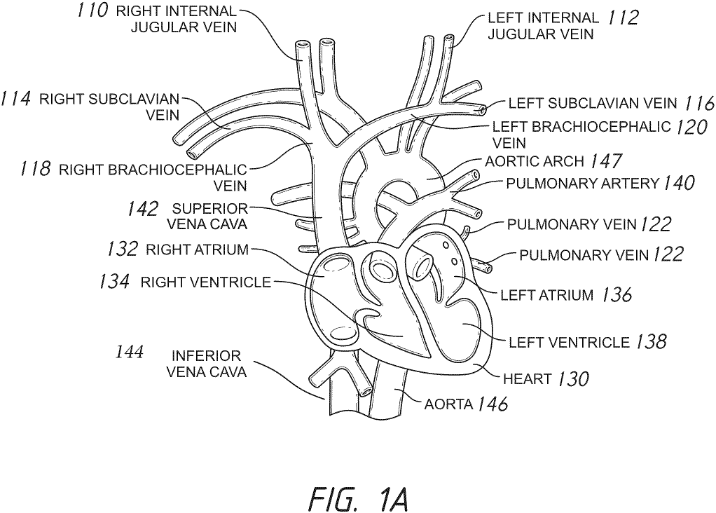

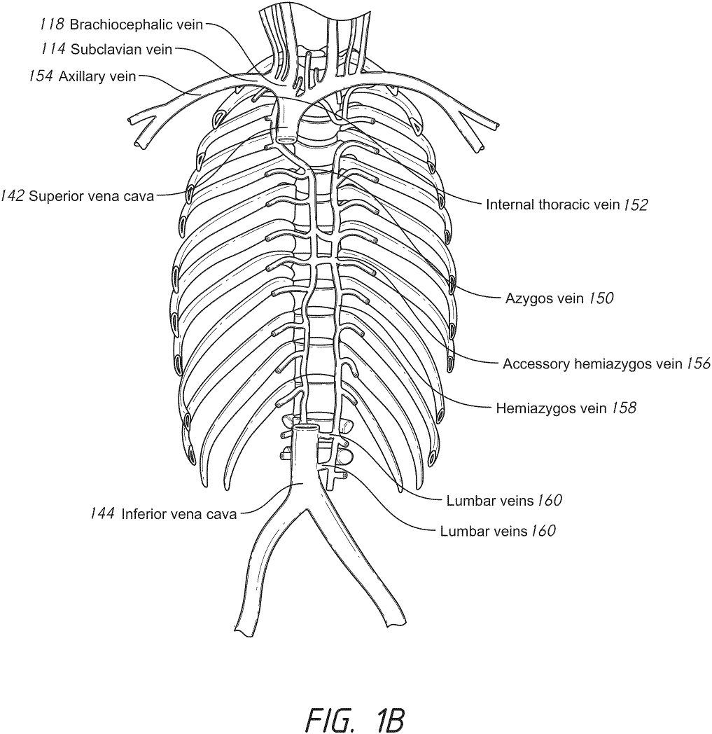

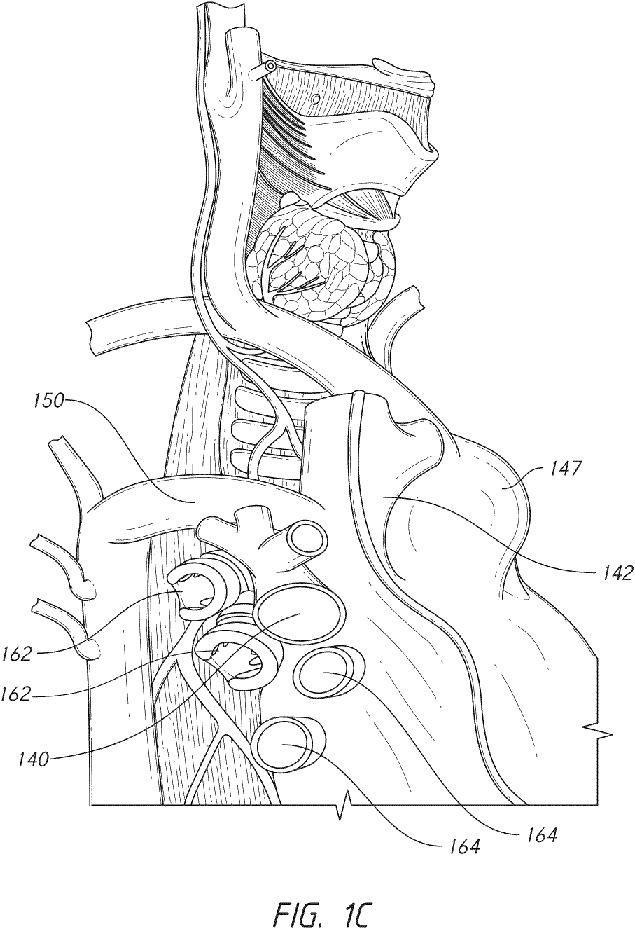

FIG. 1A illustrates a patient’s anatomy including the heart 130 with the right atrium 132, the right ventricle 134, the pulmonary artery 140, the pulmonary veins 122, the left atrium 136, the left ventricle 138, and the aorta 146 including the aortic arch 147. The inferior vena cava 144 and superior vena cava 142 with the left internal jugular vein 112 and right internal jugular vein 110, left subclavian vein 116 and right subclavian vein 114, and left brachiocephalic vein 120 and right brachiocephalic vein 118 are also shown. This central cardiovascular complex collects deoxygenated blood from the body, pumps it to the lungs and back for oxygenation, and then pumps oxygenated blood to the body.

FIG. 1B illustrates a patient’s venous anatomy in the chest, including the azygos vein 150 which flows into the superior vena cava 142, along with the accessory hemiazygos vein 156, the hemiazygos vein 158, the lumbar veins 160, and other vessels that flow into the azygos vein 150. Also shown are the right brachiocephalic vein 118, right subclavian vein 114, axillary vein 154, and the internal thoracic vein 152 that also flow into the superior vena cava 142.

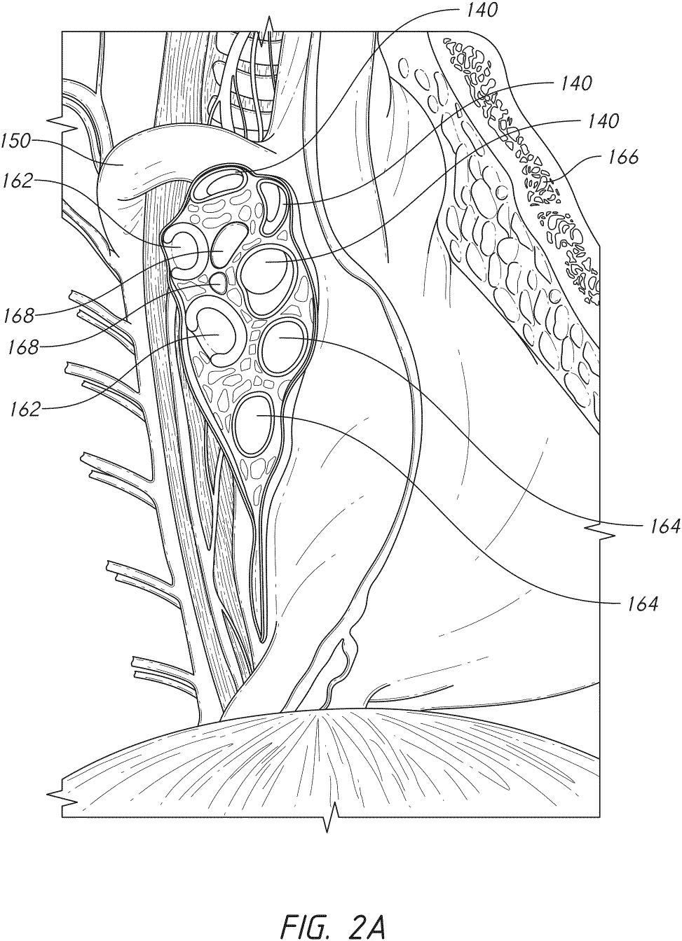

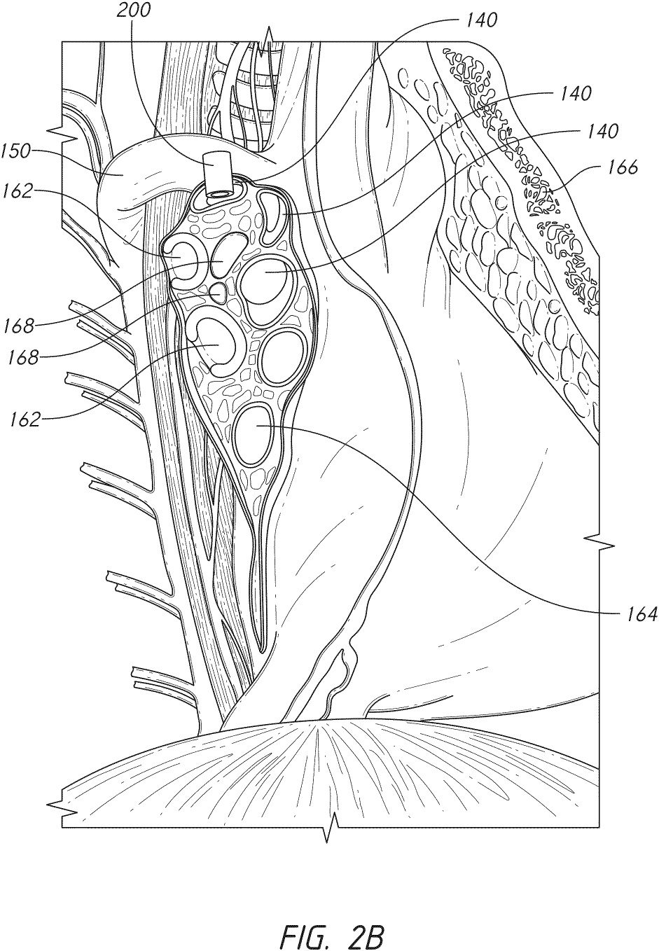

FIGS. 1C, 2A, and 2B illustrate the upper chest, including the sternum 166, right bronchus 162, right pulmonary veins 164, and lymph nodes 168. As shown in FIG. 2B, branches of the right pulmonary artery 140 extend adjacent the azygos vein 150, which provides for a possible location for implantation of a shunt 200. In some implementations, any branch of the pulmonary artery 140 may be suitable. For example, locations distal to the right pulmonary artery, including the truncus anterior, right superior trunk, apical artery, anterior artery, posterior recurrent artery, various intersegmentary branches, ascending artery, and interlobar artery may be suitable locations. Similarly, although the proximal arched section of the azygos vein 150 that extends over the pulmonary artery 140 just before the junction with the superior vena cava 142 is shown, any location along the azygos vein 150 may be suitable. Implant location may be chosen based on the patient’s particular anatomy, the distance between the branch of the pulmonary artery 140 and the azygos vein 150, the estimated blood flow through the respective vessels, estimated pressure differentials, the ease of surgical access, avoidance of any intervening structures (such as right bronchus 162, lymph nodes 168, nerves, and other non-target vasculature), and/or the preference of the implanting physician, among other factors.

A device, such as shunt 200, implanted at this location in some implementations is suitable for redirecting or diverting an amount of blood from the right pulmonary artery 140 that is leaving the right ventricle 134 of the heart 130 into the azygos vein 150 that passes adjacent to the right pulmonary artery 140. In some implementations, shunt 200, shown within both the pulmonary artery 140 and azygos vein 150 for clarity in FIG. 2B, may form a passage between the arterial and venous systems to help create a splanchnic compartment in the thoracic vessels. When the right pulmonary artery 140 is connected to the azygos vein 150, the pressure differential between the arterial and venous systems may cause blood to flow from the pulmonary artery 140 into the azygos vein 140, and further may create backflow in the azygos vein 150. An implant such as the shunt 200 may be delivered percutaneously into a patient in a collapsed configuration and expanded to an expanded configuration upon implantation. The blood that is shunted from the right pulmonary artery 140 into the azygos vein 150 may cause the intercostal veins to grow larger (dilate) and pressurize, which effectively increases intravascular volume and creates a tank of extra blood within the intercostal veins. The redirecting or diverting of blood may be sufficiently accommodated in a venous capacitance system. The redirecting or diverting of blood may result in increasing venous capacitance to increase cardiac output. At least some of the blood redirected into the azygos vein 150 can continue into the superior vena cava 142 and back to the right atrium 132, or it may flow through other blood vessels, e.g., the accessory hemiazygos vein 156, the hemiazygos vein 158 and the internal mammary vein (or internal thoracic vein 152), to the superior vena cava 142 or the inferior vena cava 144 and into the right atrium 132. The flow circuit that is created from the right pulmonary artery 140, through the azygos vein 150 and back into the right atrium 132 effectively forms the tank of extra blood in a splanchnic compartment within the body.

In some implementations, the amount of blood that is redirected to the azygos vein 150, and therefore the amount of increased venous capacitance or volume of the extra tank of blood, can be controlled by selecting an appropriate diameter for the implant, such as shunt 200. In some embodiments, a control mechanism (e.g., a controllable fluid restrictor 1000 discussed below) can be incorporated into the shunt 200 to control the flow of blood into the azygos vein 150. It is expected that the volume increase by the blood flow from the azygos vein 150 to the superior vena cava 142 may be to a hemodynamically significant degree, in some examples at least about 0.5 L or 1 L or more.

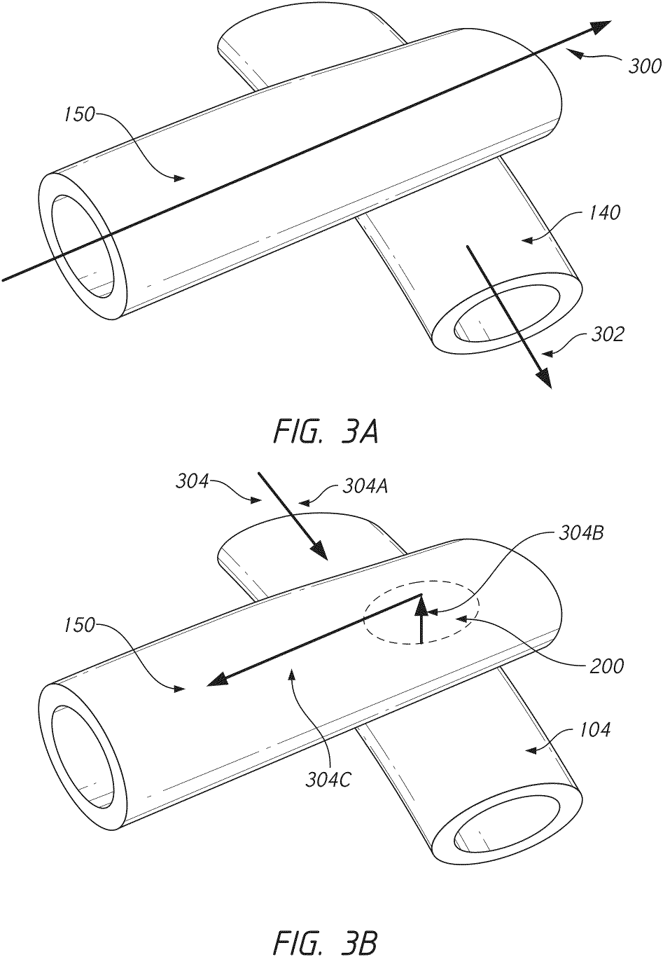

As illustrated in FIG. 3A, the azygos vein 150 normally flows in a forward or antegrade direction 300 into the superior vena cava 142. The pulmonary artery 140 flows in a forward or antegrade direction 302 to the lungs. As illustrated in FIG. 3B, creation of a passage or fistula 200 between the pulmonary artery 140 and the azygos vein may cause a diversion or redirection of blood flow in the ayzgos vein. The passage or fistula 200 may be provided by any of the implants as described herein, or may be created by other devices or techniques. In some implementations, after creation of the passage or fistula 200, blood from the right ventricle 134 flows through the pulmonary artery 140 along forward or antegrade direction 304A, as normal. Blood may then be redirected along direction 304B, through passage 200, and into the azygos vein 150. From the azygos vein 150, at least some of the blood may be redirected to flow in a reverse or retrograde direction 304C within the azygos vein, back toward the thoracic cavity. In some implementations, all the blood is diverted to reverse or retrograde direction 304C. In some implementations, some blood continues in forward or antegrade direction 300, into the superior vena cava 142. In some implementations, the connection between the azygos vein 150 and the superior vena cava 142 may be closed off or controlled or regulated (e.g., surgically or with a valve), to direct the blood that has been diverted from the right pulmonary artery 140 into the accessory hemiazygos vein 156, the hemiazygos vein 158, the internal thoracic veins 152, or other vessels including the intercostal veins, the internal mammary veins, and others as described above.

The amount of blood that is diverted from the right pulmonary artery 140 into the azygos vein 150 reduces the amount of blood that reaches the lungs, which may advantageously reduce the stress and decongest the lungs and reduce left ventricular end diastolic pressure (LVEDP). The diverting of blood may be sufficient to reduce pulmonary artery pressure to relieve pulmonary hypertension and consequently reduce a workload of a right ventricle 134 of the patient. Treatment can be tailored by controlling one or both of the amount of blood diverted from the right pulmonary artery 140 and the amount of blood that flows from the azygos vein 150 to the superior vena cava 142.

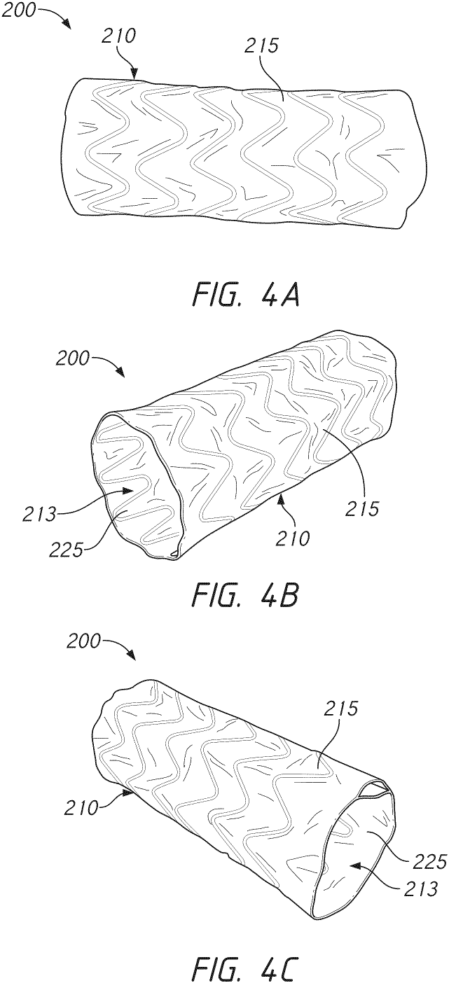

The connection between the right pulmonary artery 140 (or branch of the right pulmonary artery 140) can be created surgically or percutaneously by placing a device, such as shunt 200 between the right pulmonary artery 140 and the azygos vein 150. For example, FIG. 4A shows a side view, FIG. 4B shows a perspective view, and FIG. 4C shows another perspective view of a shunt 200. The implant or shunt 200 can include an expandable body 210 having lumen 213 between two ends. In some implementations, a first end of the shunt is positioned in the pulmonary artery, a second end of the shunt is positioned in the azygos vein, and a middle portion of the shunt is positioned at a connection between the pulmonary artery and the azygos vein. The expandable body 210 can be configured to collapse for delivery into the patient and expand into engagement with an inner wall of one or more vessels of the patient once implanted, with the expanded configuration shown. For example, a first or upstream portion of the shunt 200 may radially expand along and engage with a length of the pulmonary artery 140 upstream of the connection between the pulmonary artery 140 and the azygos vein 150, with respect to a direction of blood flow in the pulmonary artery. A second or downstream portion of the shunt 200 may radially expand along and engage with a length of the azygos vein 150 upstream of the connection between the pulmonary artery 150 and the azygos vein 140, with respect to a direction of blood flow in the azygos vein. Once implanted, blood flowing through the vessel(s) in which the implant 200 is implanted can flow through the lumen 213.

As shown, shunt 200 may include an inner body 225 located within an outer body 215. In some implementations, an implant such as shunt 200 may include an expandable body 210 with an outer body 215 of an expandable membrane or fabric layer at least partially covering an inner body 225 of a metallic frame comprising one or more struts as discussed above. In some implementations, shunt 200 comprising an inner body 225 made of an expandable tubular frame (e.g., nitinol) that may comprise struts. The struts may be covered with an outer body 215, such as a membrane or fabric of biocompatible material, for example PTFE. In some implementations, the expandable body 210 can include an expandable membrane or fabric inner body 225 surrounded or partially surrounded by a tubular outer body 215 of an expandable stent-like metallic frame as described above.

In some implementations, shunt 200 has a length sufficient to connect the pulmonary artery 140 to the azygos vein 150. In some implementations, each end of shunt 200 may include a flare, barbs, hooks, or other anchor structures to help secure the end in a respective vessel. In some implementations, shunt 200 may be tubular and have a sufficient length to radially expand against and extend along a length of the pulmonary artery 140 to secure an upstream or entrance end of the shunt 200 and direct blood from the pulmonary artery 140 into the shunt 200. The length may also be sufficient to radially expand against and extend along a length of the azygos vein 150, for example to secure a downstream or exit end of the shunt 200 and direct blood into the azygos vein 150. In some implementations, the downstream or exit end of shunt 200 is configured to direct blood upstream in the azygos vein 150, toward the thoracic vessels and splanchnic cavity described above. In some implementations, the downstream or exit end of shunt 200 is configured to direct blood downstream in the azygos vein 150, toward the superior vena cava 142.

In some implementations, the expandable body 210 can be configured as a double-walled stent, with the outer body 215 comprising the outer wall, and the inner body 225 comprising the inner wall. Each of the outer body 215 and inner body 225 can include a material layer. Each of the outer body 215 and the inner body 225 can include frames comprising a plurality of struts. In some implementations, one or more struts are sandwiched between the outer body 215 and the inner body 225. In some implementations, the plurality of struts, for example struts of the outer body 215, struts of the inner body 225, or struts between the outer body 215 and the outer body 225, are made of a single wire. In some implementations, the plurality of struts may be cut from one or more tubes.

Both the outer body 215 and the inner body 225 can be configured to collapse and expand as described herein. Additionally, the outer body 215 and the inner body 225 can be configured to collapse and expand together. For example, in some implementations, the outer body 215 and inner body 225 are configured to be implanted together and expand together within the target vessels. In some implementations, the outer body 215 and inner body 225 are configured to be implanted separately or serially. For example, outer body 215 may be implanted and expanded first, then inner body 225 may be implanted and expanded within the outer body 215.

In some implementations, one or both of the outer body 215 and inner body 225 may have porous sections, as discussed below. In some implementations, the outer body 215 and inner body 225 may have the same length and aligned proximal and distal ends, as shown in FIGS. 4A-C, to create a double-walled stent as discussed above. In some implementations, the outer body 215 and inner body 225 may have different lengths and/or may have proximal and/or distal ends that are not aligned, thereby creating sections of stent 200 with one wall and sections with two walls. In some implementations, the outer body 215 and inner body 225 may have sections. For example, stent 200 may include an outer body 215 of a material, and an inner body 225 with a first metallic strut section, for example at one end, and a second metallic strut section, for example at the other end. In some implementations, the implanted device may include multiple devices 200 as discussed below.

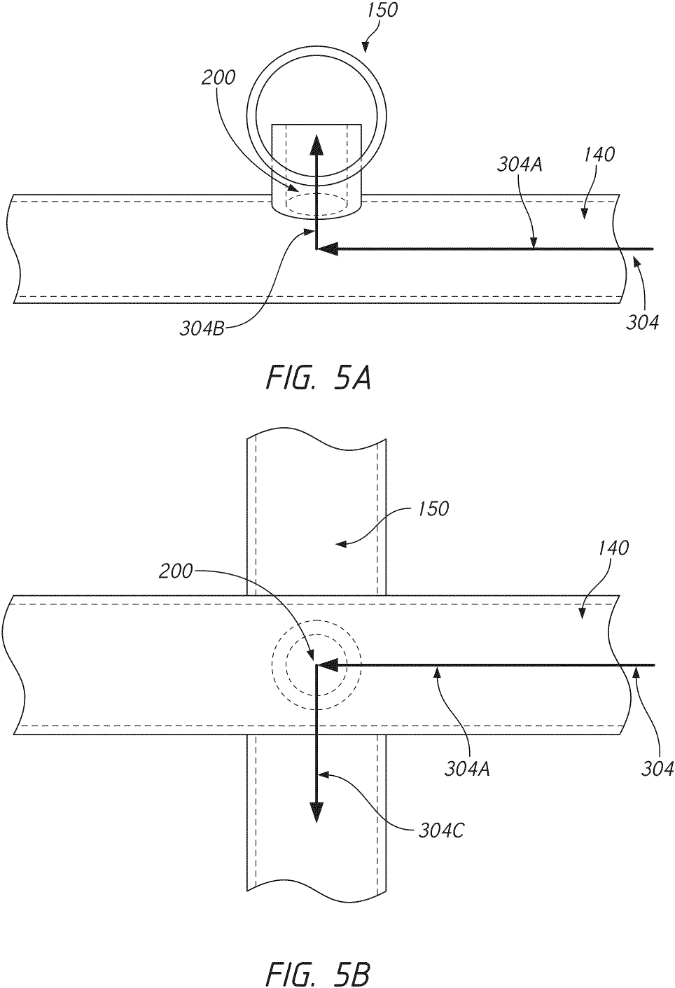

In some implementations, such as the example shown in FIGS. 5A-B, the implant may be a short shunt 200 configured to provide a connection between two vessels (e.g., between the pulmonary artery 140 and the azygos vein 150). This shunt 200 may preferably be positioned between the pulmonary artery 140 and the azygos vein 150 without extending substantially into the lumen of either vessel. As illustrated in side view FIG. 5A, the shunt 200 may be implanted to create a flow pathway between a branch of the pulmonary artery 140 and the azygos vein 150. This location allows blood flow 304 to continue along forward or antegrade direction 304A within the branch of the pulmonary artery 140 toward the lung, as normal, and a portion of the blood flow 304 to be diverted through the shunt 200 along direction 304B into the azygos vein 150. As illustrated in top view FIG. 5B, blood flow 304 exits the shunt 200 into the azygos vein 150 and may provide backflow along reverse or retrograde direction 304C. Such backflow may create a thoracic tank in the chest vessels, such as intercostals, the accessory hemiazygos vein 156, the hemiazygos vein 158, and others as discussed above. A portion of the diverted blood may also enter the superior vena cava 142. As described further below, in some implementations the shunt 200 may comprise a controllable valve. Also as described further below, in some implementations blood flow from the azygos vein 150 to the superior vena cava 142 may be separately restricted or occluded, such as by positioning an additional flow restricting implant within the azygos vein 150 downstream of the shunt 200.

Diversion of blood in the azygos vein 150 in this and other implementations may advantageously create a thoracic tank that can mimic a splanchnic vascular capacitance and redistribute blood into a splanchnic compartment, thereby offloading the heart. Reducing pulmonary artery pressure can relieve pulmonary hypertension and consequently reduce a workload of a right ventricle. Diverting the blood flow may also decongest lungs of the patient and reduce a left ventricular end diastolic pressure (LVEDP).

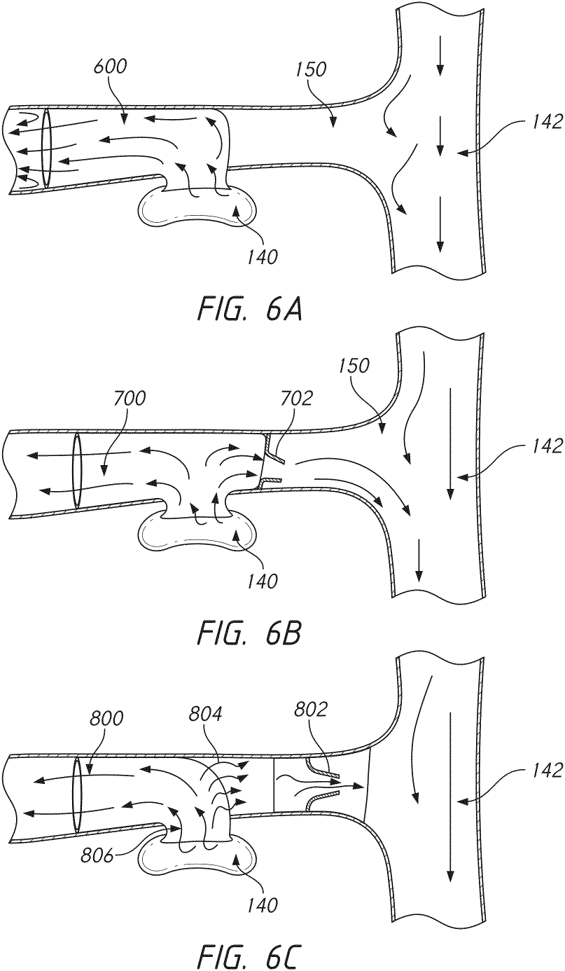

FIGS. 6A-C illustrate additional implementations of an implanted device to redirect blood flow in the azygos vein 150. In some implementations, one end of implant 600 is located within the lumen of a branch of a pulmonary artery 140 and another end of implant 600 is located within the lumen of the azygos vein 150 as illustrated in FIG. 6A. In this example, one or both ends of the implant 600 is directed within the respective vessel to further assist the desired resulting blood flow. As shown, the end of implant 600 within the azygos vein 150 is placed upstream of the implant location with respect to the direction of blood flow in the azygos vein 150 to create backflow and redirect blood from the pulmonary artery 140 into the thoracic tank as described above. Similarly, the end of implant 600 within the pulmonary artery 140 may be directed upstream to collect blood flowing in the pulmonary artery 140. In some implementations, implant 600 may be a longer shunt, that may be otherwise identical to shunt 200 discussed above. In other implementations, the upstream end of the implant 600 may extend into the pulmonary artery 140 without substantially obstructing the pulmonary artery 140, to allow blood to flow into the implant 600 as well as to continue downstream through the pulmonary artery 140. The implant 600 may be non-porous or include one or more non-porous sections to direct, limit, or prohibit blood flow between the azygos vein 150 and the superior vena cava 142.

In some implementations, an implant 700 may include one or more valves 702. For example, as shown in FIG. 6B, one end of implant 700 is located within the pulmonary artery 140 and another end of implant 700 is located within the azygos vein 150 upstream of the new connection between the pulmonary artery 140 and the azygos vein 150. Implant 700 may be similar to shunt 200 or implant 600 in some respects. A valve 702 may be included in the implant 700 to selectively direct blood from the pulmonary artery 140 to the azygos vein 150 and/or the superior vena cava 142. For example, valve 702 may be located along the length of the implant 700 to selectively allow blood from the pulmonary artery 140 in a forward direction in the azygos vein 150 into the superior vena cava 142. The valve 702 may be located on a portion of the implant 700 that is downstream from the connection between the pulmonary artery 140 and the azygos vein 150. In some implementations, valve 702 may be a pressure valve designed to open after a desired amount of blood has been diverted to the thoracic tank. For example, in some implementations, valve 702 is a check valve, such as a leaflet or pop valve designed to open under a predetermined pressure. In some implementations, valve 702 may be an aperture or iris designed to permit a predetermined volume of blood from the pulmonary artery 140 into the superior vena cava 142. As illustrated in FIG. 6B, in some implementations valve 702 may be located within the azygos vein 150 after implantation of the device 700. In some implementations, valve 702 may be located on a side of the implant 700 closer to the superior vena cava 142, as shown in FIG. 6B. In some implementations, valve 702 may be a one-way valve to control the direction of blood flow. In some implementations, valve 702 may be located in the azygos vein 150 closer to the thoracic tank, for example at the end of the implant 700 located within the azygos vein 150 upstream of the connection between the pulmonary artery 140 and the azygos vein 150. In some implementations, valve 702 may be located within the pulmonary artery 140 to control an amount of blood diverted from the pulmonary artery 140 through the implant 700 and into the azygos vein 150. In some implementations, valve 702 may be configured to be positioned within the implant 700 between the pulmonary artery 140 and the azygos vein 150 after implantation. In some embodiments, multiple valves 702 (e.g., two valves or three valves) may be positioned along the implant 700 at any of the locations described above.

In some implementations, an implant 800 may include a porous section 804. For example, as shown in FIG. 6C, one end of implant 800 is located within the pulmonary artery 140 and another end of implant 800 is located within the azygos vein 150. Implant 800 may be similar to shunt 200 or implants 600 and 700 in some respects. A porous section 804 may be included in at least a portion the implant 800 positioned within the azygos vein 150 to selectively direct blood from the pulmonary artery 140 to the azygos vein 150 and/or the superior vena cava 142. For example, porous section 804 may be located along the length of the implant 800 to allow blood from the pulmonary artery 140 into the superior vena cava 142. As illustrated in FIG. 6C, in some implementations porous section 804 may be located within the azygos vein 150 after implantation of the device 700. In some implementations, porous section 804 may be located on a side of the implant 800 closer to the superior vena cava 142, as shown in FIG. 6C. In some implementations, porous section 804 may be located within the pulmonary artery 140 to control an amount of blood diverted from the pulmonary artery 140 through the implant 800 and into the azygos vein 150. In some implementations, an implant 800 may include one or more porous sections 804 and one or more valves 802. Valve 802 may be similar to valve 702 in some or all respects. In some implementations, a single opening may be used in addition to or instead of porous section 804. In some implementations, an opening or porous section may be configured to allow a predetermined flow rate, pressure, and/or volume of blood to leave the implant in a particular direction.

In some implementations, an implant, for example implant 800 or similar, may be implanted to locate one end of the implant 800 in the pulmonary artery 140 as discussed above, and another end of the implant 800 within the azygos vein 150 such that the other end is arranged to radially expand against the azygos vein 150 at a location downstream from the connection between the pulmonary artery 140 and the azygos vein 150. For example, porous section 804 may be located in the azygos vein 150 closer to the thoracic tank, for example at a side of the implant 800 located within the azygos vein 150.

In some implementations, blood may be redirected upstream in the azygos vein 150 by a valve, such as valve 702, 802, and/or the controllable valves as discussed below, placed in the azygos vein 150. In some implementations, blood flow from the azygos vein 150 to the superior vena cava 142 is restricted to slow or block the exit of blood. As blood continues to enter the thoracic tank from the left ventricle 138 as normal, the restricted outflow increases the blood volume in the splanchnic compartment. In some implementations, blood flow from the azygos vein 150 to the superior vena cava 142 may be restricted or redirected upstream with an external ligature, an occluder, or other implanted device. In some implementations, the splanchnic compartment or thoracic tank may be created by restricting or preventing blood from flowing through another of the thoracic veins, for example, the accessory hemiazygos vein 156, the hemiazygos vein 158, the lumbar veins 160, and other vessels that flow into the azygos vein 150.

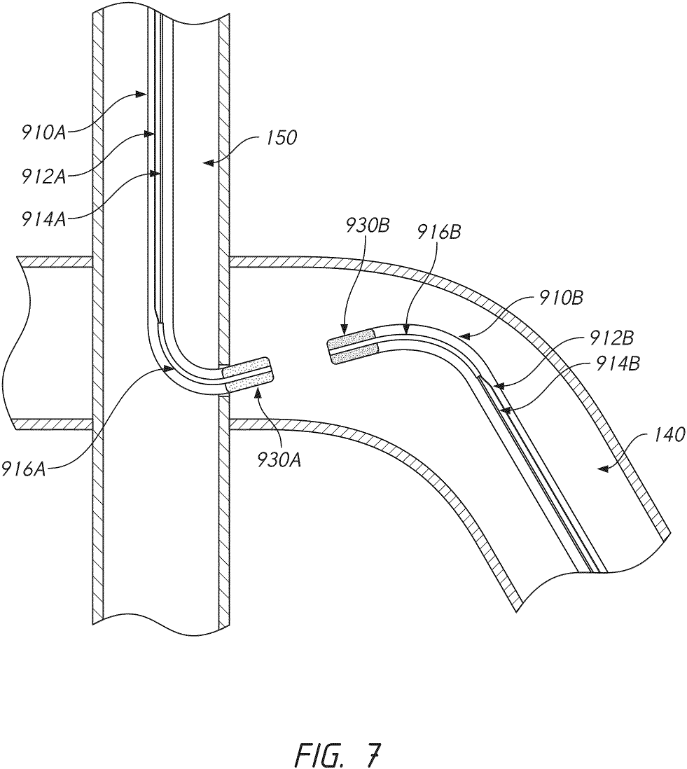

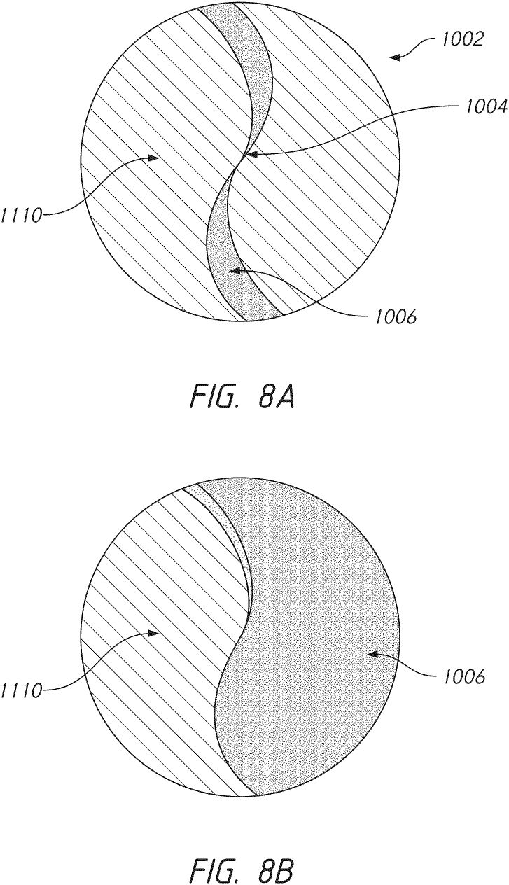

In some implementations, the implanted devices may be installed via percutaneous approach. As shown in FIG. 7, in one example of a percutaneous approach, magnetic elements may be utilized to orient two adjacent vessels. For example, in some implementations, magnetic elements may be aligned magnets, e.g., magnetic rings 930A and 930B, in each vessel. For example, a first catheter 910B may be delivered into the right pulmonary artery 140, and a second catheter 910A may be delivered into the azygos vein 150, each catheter carrying a magnet 930A, 930B. In some implementations, one or both of the catheters 910A, 910B may include an articulated section 916A, 916B or may otherwise be articulatable. In some implementations, articulated sections 916A, 916B are controlled with steering wire 912A, 912B. In some implementations, one or both catheters 910A, 910B may include pre-formed curvatures or sections that assume a pre-formed shaped. For example, in some implementations, a catheter such as catheter 910A may include as section that assumes a pre-formed shape after being released from a delivery sheath, after removal of a delivery stylet, after reaching a certain temperature, and/or after receiving an electrical pulse. In some implementations, one or both catheters 910A, 910B may comprise one or multiple lumens.

In some implementations, the magnets 930A, 930B may be used to align the catheters 910A, 910B to facilitate delivery of a guidewire (not shown) from one vessel to the other. The guidewire may be used to enable access for a delivery device used to deliver the implant. In some implementations, the magnets 930A, 930B may be connected to control wires 914A, 914B. In some implementations, control wires 914A, 914B may be used to switch a polarity of the magnets 930A, 930B to selectively repel, which may be useful for removal of the catheters 930A, 930B after delivery. In some implementations, magnets 930A, 930B may be electromagnets, and control wires 914A, 914B may be used to selectively activate and deactivate the magnets 930A, 930B at different times during placement, delivery and/or removal of the catheters 910A, 910B.

In some implementations, the azygos vein 150 may be accessed via the superior vena cava 142. In some implementations, the azygos vein 150 may be accessed via the inferior vena cava 144. In some implementations, a Swan-Ganz approach may be used to access the pulmonary artery 140. For example, the pulmonary artery 140 may be accessed via the superior vena cava 142 or the inferior vena cava 144, through the right atrium 132 and the right ventricle 134, and into the pulmonary artery 140. In some implementations, the azygos vein 150 and pulmonary artery 140 are accessed separately, as described above, and each end of the implant may be separately deployed and/or placed via tools (e.g., catheters 910A and 910B) in each vessel. In some implementations, the pulmonary artery 140 and azygos vein 150 are accessed from one vessel (e.g., a Swan-Ganz approach through the pulmonary artery 140 is used to create an opening to the azygos vein 150, or the reverse), and an implant may placed via tools in the access vessel.

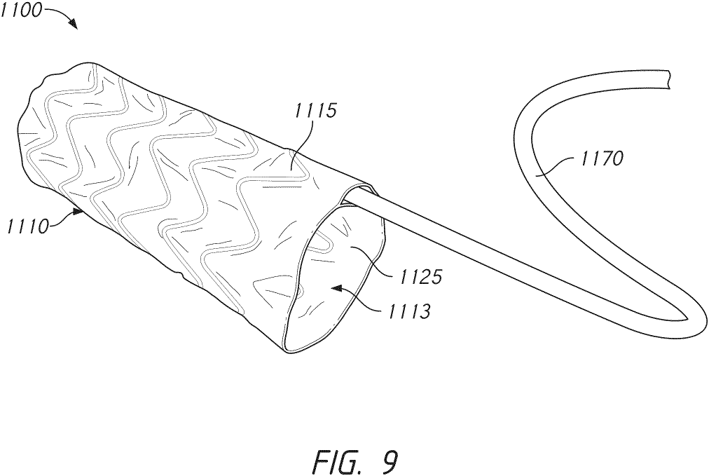

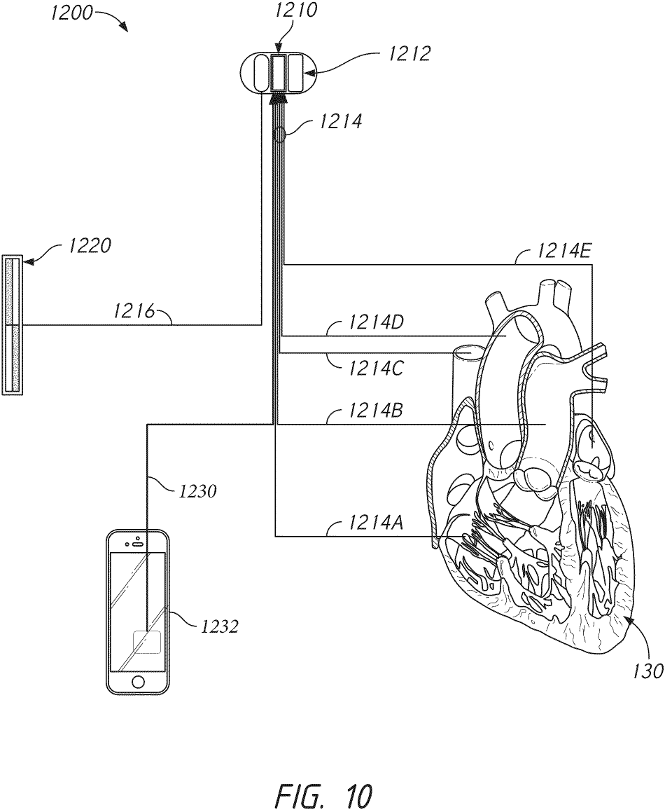

As noted above, in some implementations the implant includes a valve, for example valves 702 and 802. In some implementations, the implant includes a controllable valve or fluid restrictor to form an adjustable shunt. In some implementations, an adjustable shunt implant, for example implant 1100 shown in FIG. 9, may include a controllable fluid restrictor 1000 as shown in FIGS. 8A-8B. Adjustable shunt 1100 may be collapsible so that it may be delivered percutaneously. In some implementations, controllable fluid restrictor 1000 includes one or more wire-form frames 1002. The valve or regulator, for example controllable fluid restrictor 1000, may be located within the implant, for example adjustable shunt 1100, such that the wire-form frames 1002 extend across a cross-section of the adjustable shunt 1100. The wire-form frames 1002 may be collapsible with the rest of the adjustable shunt 1100 for delivery. When expanded, the wire-form frames 1002 form a pair of disks each having a fabric extending across part of the cross-section of the shunt 1100, as illustrated in FIGS. 8A-B.

One or more wire-form frames 1002 may be fixed or stationary with respect to the rest of the shunt 1100, for example fixed partition 1010, to prevent blood flow through a section of the controllable fluid restrictor 1000. At least one wire-form frame, for example rotating partition 1004, may rotate relative to the fabric of fixed partition 1010 to control the size of the open section 1006 through the shunt 1100. Rotating partition 1004 may also include a fabric portion prevent blood flow through the controllable fluid restrictor 1000. In some implementations, rotating partition 1004 can selectively rotate to open or close the controllable fluid restrictor 1000. As illustrated in FIG. 8A, fixed partition 1010 and rotating partition 1004 can be oriented to arrange their respective fabric portions to occlude most of the lumen or cross-section of the shunt 1100, allowing blood to pass only through a small open section 1006. In some implementations, fixed partition 1010 and rotating partition 1004 can be oriented to arrange their respective fabric portions to completely occlude the cross-section of the shunt 1100. As shown in FIG. 8B, rotating partition 1004 can be rotated to partially or completely align the fabric of the rotating partition 1004 with the fabric of the fixed partition 1010, selectively opening the controllable fluid restrictor 1000 and increasing the open section 1006 to allow more blood to pass through the controllable fluid restrictor 1000. In some implementations, the fabric of the fixed partition 1010 has approximately the same area as the fabric of the rotating partition 1004. In some implementations, the respective fabric areas of the fixed partition 1010 and the rotating partition 1004 are equal. In some implementations, the respective fabric areas of the fixed partition 1010 and the rotating partition 1004 can be arranged to occlude an area of the cross-section of the shunt 1100 that is less than the full cross-section, for example, a combined area of 95%, 90%, 80%, 75%, 60%, 50%, 45%, 33%, 20%, 15%, 10%, 5% or any other portion.

The rotation of the rotating partition 1004 may be accomplished by the drive line, for example cable 1170 shown in FIG. 9, which may comprise a torque cable driven by a motor located in a control unit. In other respects, adjustable shunt 1100 may be similar to shunt 200 and implants 600, 700, and 800, including expandable body 1110 with inner body 1125, outer body 1115, and lumen 1113. Adjustable shunt 1100 may also include additional valves and/or porous sections as discussed above.

In some embodiments, a feedback-controlled implant, such as an adjustable shunt 1100 that is positioned between the right pulmonary artery 140 and azygos vein 150, may be provided. The feedback-controlled implant may also be placed at other locations in the body, such as a shunt between two vessels or as an implant within a vessel. The implant may comprise any sort of valve or flow regulator to adjust a flow opening within the implant, and may incorporate a feedback control loop as described below. An implant as described above or elsewhere in this specification may be chronically implanted.

A system comprising an adjustable shunt according to one implementation is shown in FIG. 10. An adjustable shunt 1220 may be controlled by a drive line 1216 connected to a motor positioned in a control unit 1210 located outside of the patient or implanted. The control unit 1210 may be patient-controlled and/or patient-monitored, e.g., wirelessly through an app on a smart phone 1232. The connection between the smart phone 1232 or other suitable device and the control unit 1210 may be wireless 1230 as shown in FIG. 10. In some implementations, the connection is wired, for example when the control unit 1210 is located outside of the patient. The control unit 1210 may comprise a battery 1212 or other power source and circuitry configured to receive wired or wireless signals, for example signals 1214, from sensors. For example, pressure, flow, and/or electrical sensors may be positioned in various locations within or around the heart 130 or at other locations in the body. For example, one or more sensors may be used to measure pressure in the right ventricle 1214A, central venous pressure 1214B, pulmonary artery pressure 1214C, aortic pressure 1214D, and left atrial pressure 1214E. Based on these pressure readings and/or other signals 1214, the control unit 1210 can appropriately actuate the drive line 1216 to control the adjustable shunt 1220 in order to control the amount of blood flowing through the adjustable shunt 1220. The control logic, including the feedback loop described above, may be optimized to treat heart failure patients, for example by controlling the amount of blood that is diverted from the right pulmonary artery 140 into the azygos vein 150. This may reduce the amount of blood that reaches the lungs, which may advantageously reduce the stress and decongest the lungs and reduce left ventricular end diastolic pressure (LVEDP).

In some implementations, the adjustable shunt 1220 with controllable valve 1100 is used to tune the implant for a particular patient. For example, the rotating partition 1004 is rotated by a clinician adjust the open section 1006 via drive line 1216, and then set for a period. In such cases, the system may provide signals 1214 collected by the sensors to the clinician.

In some implementations, the shunt or any of the implants described above may comprise other mechanisms for restricting and/or occluding blood flow through the shunt, such as those described in U.S. Provisional Application No. 63/336,924, filed Apr. 29, 2022 63/494,635, filed Feb. 13, 2023, and U.S. Pat. Application No. 18/300,076, Attorney Docket No. INQB.014A, filed Apr. 13, 2023, titled SYSTEMS, DEVICES, AND METHODS FOR CONTROLLABLY AND SELECTIVELY OCCLUDING, RESTRICTING, AND DIVERTING FLOW WITHIN A PATIENT’S VASCULATURE, the entireties of which are hereby incorporated by reference.

In some implementations, a method for treating heart failure includes diverting blood from a pulmonary artery 140 to the azygos vein 150. An implanted device, such as shunt 200, adjustable shunt 1100 or 1200, and implants 600, 700, and 800, may be used to divert the blood. In some implementations, the diverting of blood is sufficient to decongest lungs of the patient and reduce a left ventricular end diastolic pressure (LVEDP) and/or to reduce pulmonary artery pressure to relieve pulmonary hypertension and consequently reduce a workload of a right ventricle of the patient. In some implementations, the diverting of blood is sufficient to mimic a splanchnic vascular capacitance and redistribute blood into a splanchnic compartment of the patient. In some implementations, the diverting of blood is sufficient to cause dilation and/or increased pressure within intercostal veins of the patient.

Some of the features or advantages encompassed by one or more of the above embodiments, or other aspects of the present application, include, but are not limited, to one or more of the following:
Increasing cardiac output by pressurizing or dilating veins, e.g., the intercostal veins;
Accommodating diverted blood in a venous capacitance system;
Diversion of blood is sufficient to decrease tension and stretch on a left ventricular wall which increases the tissue levels of vaso-constrictors like norepinephrine, Angiotensin II, and certain cytokines which have a deleterious effect on the heart and the vascular system, which result in exacerbation of heart failure;
Devices and methods for placing a shunt or creating a fistula between the right pulmonary artery and the azygos vein, to redirect blood through one or more pathways back into the right atrium;
Reducing stress on the lungs and reducing LVDP by diverting blood from the pulmonary artery to reduce the amount of blood that reaches the lungs;
A valve or regulator used to control the flow of blood between two body locations, where the valve or regulator is controlled based on a feedback loop accounting for pressures taken from various locations in the body;
A valve or regulator, as incorporated into a collapsible, percutaneously-delivered shunt;
A valve or regulator, to control the flow of blood to treat or prevent heart failure;
The use of a wire-form disk that rotates relative to a stationary disk and that is controlled by a drive line to regulate blood flow;
Mimicking a splanchnic vascular capacitance and redistributing blood into a splanchnic compartment of the patient;
Diverting of blood to counteract vaso-constrictors released by the patient as a result of heart failure;
A catheter system that uses magnets to align two different catheters placed in two different vessels in different domains so that a shunt can be created between the two vessels using standard interventional techniques. Once the alignment of the vessels is accomplished and a guidewire has been passed through the vessels, aligning magnets can be rotated in such a way that the like poles of the magnet face each other thereby disconnect from each other due to the repelling force of the magnets.

In some implementations, an implant or system may provide various features in a single implant as described above. In some implementations, the features are provided in multiple cooperating implants for modular installation.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed, others may be added. For example, the actual steps or order of steps taken in the disclosed processes may differ from those shown in the figure. Depending on the embodiment, certain of the steps described above may be removed, others may be added. Furthermore, the features and attributes of the specific embodiments disclosed above may be combined in different ways to form additional embodiments, all of which fall within the scope of the present disclosure.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the described embodiments, and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Likewise the term “and/or” in reference to a list of two or more items, covers all of the following interpretations of the word: any one of the items in the list, all of the items in the list, and any combination of the items in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

What is claimed is:

1. A system for treating heart failure of a patient, comprising:
an implant configured to be positioned in an azygos vein of a patient to at least partially occlude blood flow from the azygos vein into a superior vena cava.

2. The system of claim 1, wherein the implant further comprises a controllable valve.

3. The system of claim 1, wherein the implant comprises a shunt configured to be positioned between a pulmonary artery and the azygos vein.

4. The system of claim 1, wherein the implant is configured to redirect blood in the azygos vein.

5. The system of claim 4, wherein the implant is configured to divert blood in the azygos vein into a hemiazygos vein, an accessory hemiazygos vein, or an internal mammary or internal thoracic vein.

6. The system of claim 1, wherein the implant comprises an expandable tubular body configured to extend between a pulmonary artery and the azygos vein.

7. The system of claim 6, wherein the implant comprises an upstream end that does not substantially obstruct blood flow in the pulmonary artery.

8. The system of claim 6, wherein the implant comprises an upstream end configured to radially expand against an inner wall of the pulmonary artery.

9. The system of claim 6, wherein the implant comprises a downstream end configured to radially expand against an inner wall of the azygos vein.

10. The system of claim 6, wherein the downstream end of the implant is configured to direct blood into the azygos vein opposite to a forward direction of blood flow in the azygos vein.

11. The system of claim 6, wherein the implant comprises a valve configured to regulate blood flow from the azygos vein into a superior vena cava.

12. The system of claim 6, wherein the implant comprises a porous section configured to permit blood flow from the azygos vein into a superior vena cava.

13. The system of claim 1, wherein the implant comprises a first implant configured to be positioned in the azygos vein to at least partially occlude blood flow from the azygos vein into the superior vena cava, and further comprising a second implant configured to be direct blood from a pulmonary artery into the azygos vein.

14. The system of claim 1, further comprising a controller configured to regulate blood flow through the implant.

15. The system of claim 14, wherein the controller is configured to regulate blood flow through the implant based on one or more pressure readings.

16. A method of treating heart failure of patient, comprising restricting blood from flowing from an azygous vein into a superior vena cava of the patient.

17-25. (canceled)

26. A method for treating heart failure of a patient, the method comprising diverting blood from a pulmonary artery to an azygos vein via a shunt implanted between the pulmonary artery and the azygos vein.

27-55. (canceled)
